(19) United States
(12) Patent Application Publication
(10) Pub. No.: US 2025/0339065 A1
(43) Pub. Date: Nov. 6, 2025

(54) MEASURED VOLUME TUBE SAMPLER

(71) Applicant: NOBLE HOUSE GROUP PTY, LTD., Kingston, Australian Capital Territory (AU)

(72) Inventor: Chris WHELAN, NewHall, CA (US)

(21) Appl. No.: 19/101,990

(22) PCT Filed: Aug. 8, 2023
(86) PCT No.: PCT/AU2023/050741

§ 371 Date: Feb. 7, 2025

Related U.S. Application Data
(60) Provisional application No. 63/395,891, filed on Aug. 8, 2022.

(51) Int. Cl. A61B 5/15

(52) U.S. Cl. CPC A61B 5/15003; A61B 5/150099; A61B 5/150221; A61B 5/150236; A61B 5/150244; A61B 5/150824

(57) ABSTRACT

A sampler (10) has barrel (20) and a plunger (22) within the barrel and defining a fluid receiving volume (38). An inlet (24) is in fluid communication with the fluid receiving volume (38) via a flow path and a one way valve (76) is located in the flow path and to only allow fluid to flow from the inlet (24) into the 5 fluid receiving volume (38). The plunger (22) has an outlet (46) in fluid communication with the fluid receiving volume (38) so that movement of the plunger (22) can draw fluid into fluid receiving volume (38) via the inlet (24) and expel fluid from the fluid receiving volume (38) via the outlet (46).

### FIELD OF INVENTION

This invention relates to devices that enable taking of fluid samples, such as blood, and more particularly to devices that allow multiple fluid samples to be accurately taken during one connection event. The invention has particular application to the taking of multiple blood samples from a patient during one connection event with the patient.

### BACKGROUND

For some patients in hospitals it is necessary to take blood samples relatively frequently. Conventional blood sampling methods include a simple evacuated tube that is impaled on a cannula connected, directly or indirectly, to a blood line to draw blood into the evacuated tube. The amount of blood drawn is controlled by the user removing the evacuated tube from the cannula.

Typical evacuated tubes have a nominal volume and sometimes have a marker (e.g. a window) to indicate when “enough” fluid has been collected. However, particularly with a patient in a bed, it is difficult to orient the tube vertically for blood sampling and use any volume markings, as there is no room to manipulate the sampling site and tube for accurate measurements.

Alternatively, some users draw an amount of blood with a syringe (which allows more accurate measurement) and then transfer it to a vacuum tube. This leads to contamination hazards of an open-sampling-system, and is impractical for multiple samples-either the user reconnects to collect more each time, or they need to do the sums to collect enough in one draw for the multiple tubes they want to collect. Obviously this has usability issues and is not a robust way to address overdrawing blood from patients.

Different size tubes allow different amounts of blood to be collected. However, generally, for diagnostic tests the user will, out of an abundance of caution, take too much blood. Failing to take enough blood may result in repeat draws being required, thereby compounding the overdraw scenario.

Whilst a single blood sample is not a significant amount, when many blood samples are taken together the total amount taken can become significant, particularly if the patent is paediatric, geriatric or otherwise frail and/or if multiple samples need to be taken periodically.

Accordingly, it is desirable to be able to control the amount of blood taken from a patient more easily than merely disconnecting an evacuated tube from a cannula. Further it is desirable for small amounts of blood to be more easily taken compared to simply impaling an evacuated tube on a cannula.

### SUMMARY OF THE INVENTION

In an attempt to ameliorate at least one of the disadvantages of the prior art the invention in one broad form provides a sampler comprising:
a tubular barrel having a first barrel section with an interior closed at one end and an open at the other end;
a plunger received within the first barrel section for reciprocal movement therein via the open end and including a gasket that engages and seals against the first barrel section, thereby defining a fluid receiving volume between the closed end and the gasket;
an inlet in fluid communication with the fluid receiving volume via a flow path;
a one way valve located in the flow path and configured to allow fluid to flow from the inlet into the fluid receiving volume and to prevent fluid to flow out of the fluid receiving volume through the inlet;
the plunger including an outlet in fluid communication with the fluid receiving volume, the plunger operable to draw fluid into fluid receiving volume via the inlet and expel fluid from the fluid receiving volume via the outlet.

The plunger may include a tubular portion adapted to receive an evacuated sampling tube.

The tubular portion may be open at one end and closed at the other end, with the open end adapted to receive an evacuated tube.

The outlet preferably extends into the tubular portion.

The outlet may comprise a cannula, preferably adapted for piercing a bung of an evacuated sampling tube.

In another broad form the invention provides a sampler comprising:
a tubular barrel having a first barrel section with an interior closed at one end and an open at the other end;
a plunger received within the first barrel section for reciprocal movement therein via the open end and including a gasket that engages and seals against the first barrel section, thereby defining a fluid receiving volume between the closed end and the gasket;
an inlet in fluid communication with the fluid receiving volume via a flow path;
a one way valve located in the flow path and configured to allow fluid to flow from the inlet into the fluid receiving volume and to prevent fluid to flow out of the fluid receiving volume through the inlet;
the plunger having
   a tubular portion open at one end and adapted to receive an evacuated tube;
   a cannula mounted in the plunger and being in fluid communication with the fluid receiving volume and extending in the tubular portion with the free end of the cannula facing the open end.

The first barrel section and/or the plunger may include at least one secondary sealing member that sealing engages between the plunger and the interior of the first barrel section. Preferably the at least one secondary sealing member comprises at least one O-ring. Preferably the at least one secondary sealing member is located on the first barrel section. Preferably the at least one secondary sealing member is located at or near the open end of the first barrel section.

The tubular portion may be received within the first barrel section.

The plunger may comprise first and second plunger sections.

The second plunger section may comprise the tubular portion.

The second plunger section may remain outside of the first barrel section.

Where the second plunger section remains outside of the first barrel section it may be extend transversely or radially more than the first barrel section. This allows the second plunger section to be sized to receive an evacuated tube whilst allowing the first barrel section to be reduced in size.

The barrel may have a second barrel section that receives at least part of the second plunger section.

The second barrel section may be shorter than the second plunger section, with the open end of the second plunger section spaced from the open of the second barrel section.

The second barrel section and the second plunger section need not seal against each other.

Where the plunger comprises first and second plunger sections, the interiors of the first and second plunger sections may be closed from each other.

The region of the junction of the first and second plunger sections may comprise a cannula support.

Where the barrel and plunger each comprise a portion of constant cross section, the cannula is preferably mounted directly in the end of the plunger with at least a passageway in the plunger communicating the cannula with the fluid receiving volume. However, the cannula may extend to be in direct fluid communication with the fluid receiving volume.

The one way valve is preferably normally closed and configured to open when a first predetermined pressure difference is applied across the one way valve.

Preferably the first predetermined pressure difference is greater than that available from an evacuated tube.

The sampler may be configured such that a second predetermined pressure difference between the fluid receiving volume and the outside environment will cause the plunger to move into the barrel.

Preferably the first predetermined pressure difference is greater than the second predetermined pressure difference.

The second predetermined pressure difference may be chosen so that it is less than that available from an evacuated tube, such that when the interior of the evacuated tube becomes into fluid communication with the fluid receiving volume, the pressure difference generated draws the plunger into the barrel.

Where the initial pressure difference is insufficient or, due to the evacuated tube receiving fluid, the pressure difference has reduced below the second predetermined pressure difference an additional force may be required between the barrel and plunger to initiate and/or continue movement of the plunger inwards into the barrel.

The sampler preferably has a tubular inlet portion defining the inlet and the one way valve is preferably mounted in the interior of the barrel at an inner end of the tubular inlet portion.

The inlet may be at or near the closed end of the barrel.

The sampler is preferably configured so that when the plunger is fully inserted into the barrel the volume of the fluid receiving volume is effectively zero.

The gasket is preferably configured to engage the one way valve when the plunger is fully inserted into the barrel.

The plunger may include a plunger inner end.

The plunger inner end may be closed, with the outlet comprising a first passageway therethrough.

Where the outlet comprises a cannula the cannula may be mounted in the first passageway. The cannula may comprise a separate cannula assembly, such as a conventional screw-in cannula assembly, that is mounted in the first passageway.

The gasket may be mounted on the plunger inner end.

The outlet may comprise a second passageway through the gasket. Where the outlet comprises a cannula the cannula may be mounted in the second passageway.

A closure may be located at or near the open end of the plunger whereby the tubular portion may be sealed from the outside environment. The closure is preferably a lid or cap movable between open and closed positions. Where the closure is movable between open and closed positions preferably it is mounted for movement about a hinge.

The closure may engage the barrel when the plunger is fully inserted into the barrel.

The sampler may have two different sets of volume markings, with a first set for use when drawing a first sample of fluid into the sampler and the second set for use when drawing subsequent samples of fluid into the sampler. Where the sampler has two different sets of volume markings the sampler may include markings to indicate which set to use.

### BRIEF DESCRIPTION OF THE DRAWINGS

FIG. 1 is a schematic view of a first embodiment of the invention in use.

FIG. 2 is a plan view of a first embodiment of the invention in a first state.

FIG. 3 is a side view of the first embodiment in the first state.

FIG. 4 is a plan view of the first embodiment of the invention in an extended state.

FIG. 5 is a side view of a first embodiment of the invention in an extended state.

FIG. 6 is a cross sectional view of the first embodiment (as if taken along line AA of FIG. 3) connected to a blood line.

FIG. 7 is a cross sectional view of the first embodiment (as if taken along line AA of FIG. 5) connected to a blood line, after it has been extended to draw in a blood sample.

FIG. 8 is a cross sectional view of the first embodiment about to receive an evacuated tube.

FIG. 9 is a cross sectional view of the first embodiment after an evacuated tube has been received and impaled on an internal cannula but before any blood has passed into the evacuated tube.

FIG. 10 is a cross sectional view of the first embodiment after blood has been passed into the evacuated tube.

FIG. 11 is a cross sectional view of the first embodiment after the evacuated tube has been removed from the device.

FIG. 12 is a plan view of a second embodiment of the invention in a first state.

FIG. 13 is a side view of the second embodiment in the first state.

FIG. 14 is a plan view of the second embodiment of the invention in an extended state.

FIG. 15 is a side view of a second embodiment of the invention in an extended state.

FIG. 16 is a cross sectional view of the second embodiment (as if taken along line AA of FIG. 13) connected to a blood line.

FIG. 17 is a cross sectional view of the second embodiment (as if taken along line AA of FIG. 15) connected to a blood line, after it has been extended to draw in a blood sample.

FIG. 18 is a cross sectional view of the second embodiment about to receive an evacuated tube.

FIG. 19 is a cross sectional view of the second embodiment after an evacuated tube has been received and impaled on an internal cannula but before any blood has passed into the evacuated tube.

FIG. 20 is a cross sectional view of the second embodiment after blood has passed into the evacuated tube.

FIG. 21 is a cross sectional view of the second embodiment after the evacuated tube has been removed from the device.

### DETAILED DESCRIPTION OF PREFERRED AND OTHER EMBODIMENTS

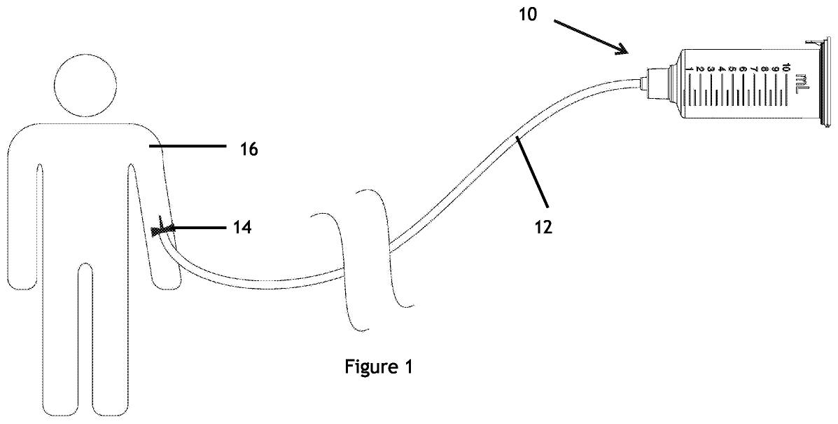

FIG. 1 schematically shows a sampler 10 according to a first embodiment of the invention attached to a blood line 12 in turn connected via a winged sampling needle 14 to a patient 16.

The sampler 10 is similar to a medical syringe and has two main components, an outer hollow tubular barrel 20 and an inner plunger 22. The barrel 20 has an inlet 24, usually with a standard male Luer fitting for connection to standardised medical fittings, at a first, upstream, end 26 and is open at its other end 28. The plunger 22 is received within the hollow interior of the barrel via the open end 28 and may move axially within the barrel 20.

A gasket 30 is mounted at or near an inner end 32 of the plunger 22 and seals between the plunger 22 and the inner wall 36 of the barrel 20. Thus the effective volume 38 of the interior of the barrel between the inlet 24 and the gasket 30 on the inner end of the plunger 22 may be varied by movement of the plunger 22.

The plunger 22 is generally tubular and has a hollow tubular section 40 with inner and outer ends 32 and 34 respectively. The tubular section 40 has a cross section complementary to and slightly smaller than that of the tubular portion 44 of the barrel 20. The barrel 20 and plunger 22 are preferably cylindrical so that the barrel 22 and plunger 20 may be located at any relative rotational position to each other.

A cannula 46 is mounted axially at the inner end 32 of the plunger 22 and extends within the interior 48 of the plunger 22 toward the open end 34 with its pointed free end 50 facing the outer end 34. The cannula 46 is preferably sheathed with a rubber or other elastomeric sheath 52.

The inner end 32 of the plunger 22 includes a passageway 54 that is in fluid communication with the bore 56 of the cannula 46. The gasket 30 has a passageway or opening 58 that is in fluid communication with the passageway 54 and so the bore 56 of the cannula 46 is in fluid communication with the interior 38 of the barrel 20. If desired the cannula 46 may extend to the outer surface 60 of the inner end 32 and communicate directly with the passageway or opening 58 in the gasket 30, or even extend through the gasket 30 to align with the outer surface 60 of the gasket 30. In the preferred embodiments the plunger 22 is injection moulded around the cannula 46 but the cannula 46 may be a separate component, such as a standard screw-in cannula assembly, which is mounted on or in the plunger 22 after the plunger 22 is manufactured.

The barrel 20 and plunger 22 are sized so that a standard size evacuated tube may pass into the interior 48 of the plunger 22 and be impaled on the cannula 46.

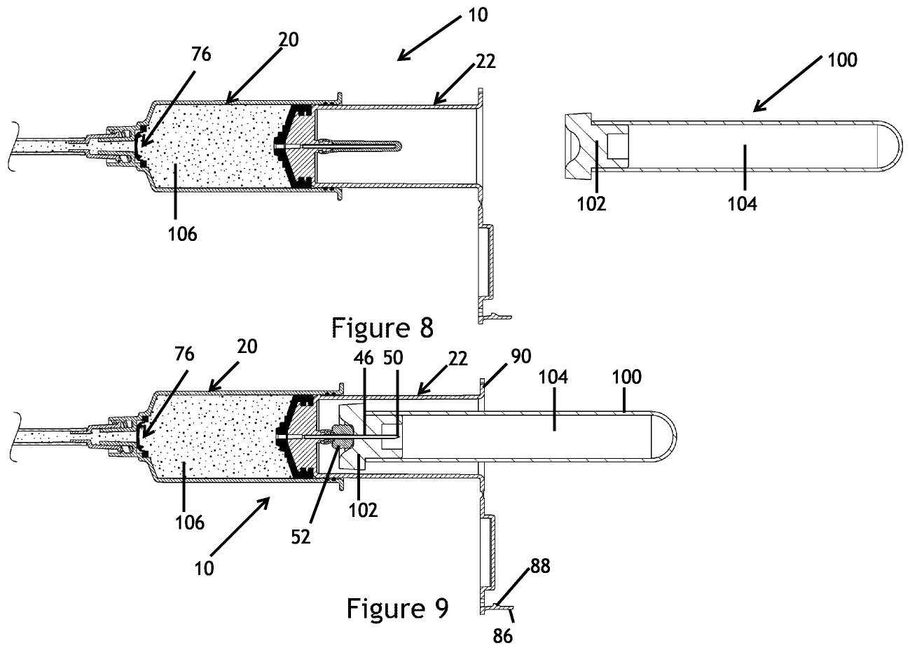

A closure 64 is provided at the outer end 34 of the plunger 22 for sealing the interior 48 of the plunger from the outside environment. The closure 64 is preferably hinged to tubular section 40 about hinge 66 so as to be movable between a closed position, as in FIG. 1, and an open position, as in FIG. 8.

In the closed position the closure 64 seals the interior 48 of the tubular section 40 from the outside environment and in the open position provides access to the interior 48. The closure 66 is preferably formed integrally with the tubular section 40 but the tubular section 40 and closure 66 may be formed as separate components and joined together post manufacture. A hinge is preferred as it allows easy opening and closing of the closure. Whilst not preferred a tether may be used instead, or even omitted.

The closure 64 is preferably a transition fit within the tubular section 40 and, accordingly, has a closed inner tubular portion 68, which extends into the tubular section 40 and engages the inner surface 70. An annular flange 72 extends around the inner portion 68 and overlays an annual flange 74 at the end of the tubular section 40.

The barrel 20 differs from a conventional syringe barrel in a number of features. A one way valve 76 is located at the end 26 and allows fluid to be drawn into the interior 38 via the inlet 24 but prevents fluid exiting the interior 38 via the inlet 24.

Two sealing rings 78 (there may be none, only one or more than two) are located near the other end 28 on the interior surface 36 of the barrel and engage the outer surface 80 of the plunger 22. Each sealing ring 78 is preferably a suitable medical grade elastomer O-ring, whether a natural (e.g. rubber) or synthetic polymer. The interior surface 36 of the barrel 20 preferably has a suitable groove 82 for location of each sealing ring. The sealing ring(s) 78 serve as a secondary barrier between the gasket 30 and the sealing ring(s) 78 and so aid to isolate the interior surface 36 of the barrel 20 from the outside environment. The sealing ring(s) 78 and any associated locating structure, such as groove(s) 82, are optional and may be omitted.

The barrel 20 has an annular flange 84 at its open end. The closure 64 includes an arm 86 that, when in the closed position, and the plunger 22 is fully inserted into the barrel 20, extends over the barrel's annular flange 84. The arm 86 has an inwardly extending protrusion 88 that engages the far side of the flange 84. The arm 86 thus holds the plunger 22 in the inserted position in the barrel 20 and also prevents inadvertent opening of the closure 64. There may be multiple arms 86 that engage the barrel flange 84. The arm 86 deflects when the plunger is withdrawn to ride over the barrel flange 84.

The barrel flange 84 is preferably circular so that the plunger 22 may be inserted at any rotational position relative to the barrel 20 and the arm(s) 86 will engage the flange 84. Whilst not preferred, the barrel 20 may be provided with one or more separate flanges with which the arm(s) 86 may engage. However, that requires the plunger to be rotationally positioned with one or more arms aligned with a respective barrel flange.

The annular flange 74 of the plunger abuts the barrel flange 84 when the plunger 22 is fully inserted into the barrel 20. The plunger flange 74 need not extend fully around the plunger and/or need not be circular. The plunger flange 74 is preferably the same diameter as the barrel flange 84 and a slot or recess 90 is provided in the plunger flange 74 for the or each arm 86. The protrusion 88 preferably extends inwards of the periphery of the plunger flange 74, so that opening of the closure requires the arm 86 to deflect to ride over the plunger flange 74.

The plunger flange 74 may have a larger diameter than the barrel flange 84 so that the plunger 22 and closure 64 may the more easily withdrawn together from the barrel 20 whilst the closure 64 remains in the closed position. In addition or alternatively the flange 74 may include two or more finger grip portions that extend outwardly more than the remainder of the flange 74.

The barrel 20 has at least one set of volume markings 92 that correspond to the effective volume 38 of the barrel 20, depending on the plunger position.

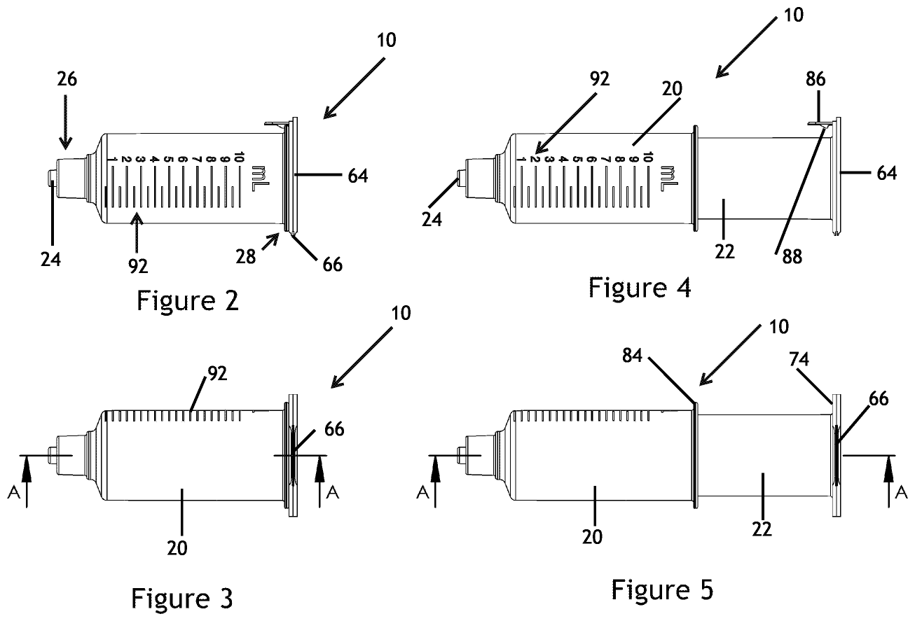

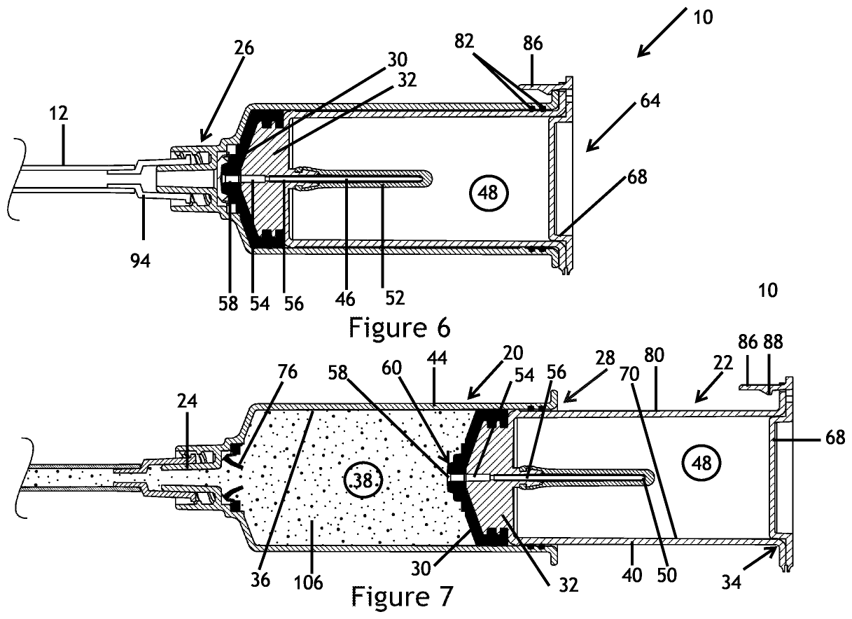

The sampler 10 is supplied sterilised and in the fully inserted and closed position, as in FIG. 2. The inlet 24 of the sampler 10 is connected to a blood line via the fitting 94, as shown in FIG. 6. The user withdraws the plunger 22 from the barrel 20, whilst leaving the closure 64 in the closed position. The reduced pressure opens the one way valve 76 and draws blood 106 into the interior 38. The plunger is withdrawn to draw the required amount of blood into the barrel 22, using the volume markings 92 for guidance, as shown in FIG. 7. The one way valve 76 is normally closed and is biased to return to the closed position. Thus once outward movement of the plunger ceases and the pressure across the one way valve 76 equalises the one way valve 76 closes, as shown in FIG. 8. The closure 64 is then opened, as in FIG. 8, providing access to the interior 48 of the plunger 22 and the cannula 46 mounted therein.

Whilst the sampler 10 remains connected to the blood line 12 the user then inserts an evacuated tube 100 into the interior 48 of the plunger 22 and impales the elastomer bung 102 of the evacuated tube 100 on the cannula 46. The evacuated tube 100 is inserted into the plunger 22 until the free end 50 of the cannula 46 has passed through the bung 102 into the interior 104 of the evacuated tube 100, as in FIG. 9.

The act of impaling the evacuated tube 100 on the cannula 46 may apply a compressive force between the plunger 22 and the barrel 20 if the user is holding the barrel 20 in one hand whilst holding the evacuated tube 100 in the other. However, whilst the cannula 46 is not in communication with the interior 104 of the evacuated tube, the one way valve 76 prevents any back flow of blood 106 out of the barrel interior 38 back into the blood line 12. Thus the plunger 22 cannot move inwards relative to the barrel 20. Until the free end 50 of the cannula 46 has passed into the interior 104 the bung 102 blocks the free end 50 of cannula and so prevents blood escaping out of the interior 38 via cannula 46. Accordingly, the volume of blood 106 in the barrel 20 remains unchanged.

Outward movement of the plunger 22 relative to the barrel 20 could result in more blood being drawn into the barrel whilst the evacuated tube is being impaled on cannula, or afterwards. This may occur by the user grasping the tubular section 40 of the plunger 22 or the plunger flange 74 whilst inserting the evacuated tube and simultaneously pulling outwards relative to the barrel, but this is unlikely. The friction provided by the gasket 30 and sealing ring(s) 78 may be selected to also reduce the risk of inadvertent movement between plunger 22 and barrel 30.

Once the free end 50 of the cannula 46 has passed into the interior 104 of the evacuated tube the interior 104 is then in fluid communication with the blood filled interior 38 of the barrel 20.

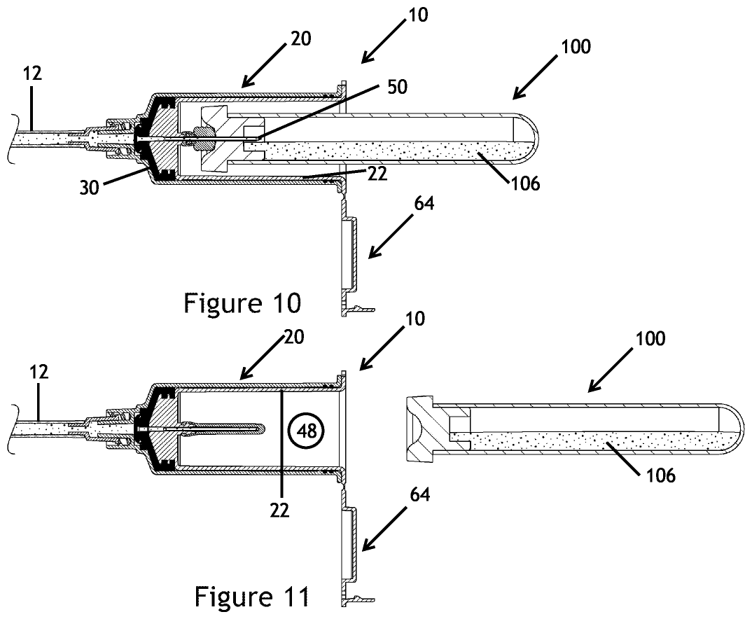

The one way valve 76 is selected so that the increase in pressure difference across the valve 76 created by the negative pressure in the evacuated tube 100 is less than that required to open the valve 76. Thus the one way valve 76 remains closed. If the valve 76 were to open due to that increased pressure difference, blood could flow into the barrel interior 38 whilst simultaneously flowing into the evacuated tube 100, so rendering any initial volume measurement meaningless. The user then manually presses the plunger 22 into the barrel 20 until the plunger 22 is fully inserted, allowing substantially all of the blood 106 in the barrel interior 38 to be transferred into the evacuated tube 104, as shown in FIG. 10. Again, the one way valve 76 prevents any back flow of blood 106 into the blood line 12. By fully inserting the plunger 22 a known quantity of blood 106 is passed into the evacuated tube 100.

The surface 60 of the gasket 30 is shaped to be complementary to the surface of the end region of the barrel and the one way valve so that when the plunger is fully inserted into the barrel there is substantially no blood between the gasket 76 and inner surface 36 of the barrel and one way valve 76.

Appropriate selection of the gasket 30 and sealing ring(s) 78 may allow the pressure difference created by the evacuated tube 100 in the barrel interior 38 to overcome the resistance to movement provided by those components whilst not opening the one way valve 76. Thus, on the cannula free end 50 entering the evacuated tube interior 104, the plunger may be drawn inwardly, preferably fully, automatically transferring the blood 106 from the barrel into the interior 104.

By preventing backflow of blood the one way valve 76 also prevents any contamination of the sampler interior 38 from passing back to the patient.

The evacuated tube 100 is withdrawn from the plunger 22 whilst the plunger 22 is maintained fully inserted into the barrel 20, as shown in FIG. 11. The procedure may then be repeated as many times as required. Where different volumes of blood are required the user merely retracts the plunger 22 to the required location on the volume marking 92.

The sampler 10 is intended for taking of multiple samples of blood within a short period of time and is not intended to remain connected after a group of samples has been taken and may be disconnected and disposed of after the group of samples has been taken.

The embodiment of FIGS. 1 to 11 is intended for taking blood samples with volumes of more than about 1 ml and is only accurate to about 0.5 ml. Typically the blood tests that require more than 1 ml do not require more than about 10 ml and accordingly the maximum volume that can be drawn into the barrel is about 10 ml. It will be appreciated that devices according to the invention may be made with different maximum volumes.

Some blood tests require significantly less blood, typically less than 2 ml and so taking significantly more blood (such as 5 to 10 ml) is wasteful. Taking smaller samples using a device intended for larger volumes is also potentially inaccurate, leading to wastage or measurement error. Where many samples are required periodically the loss of blood can impact on patient health.

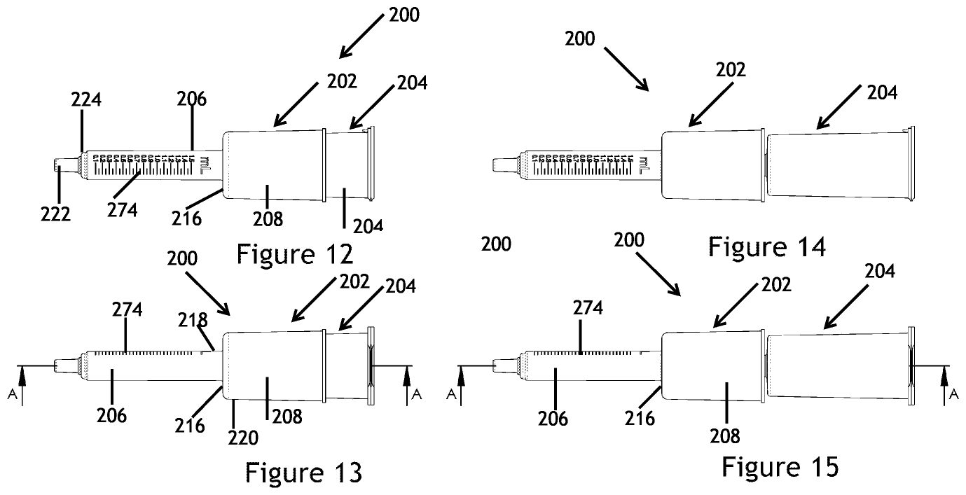

Referring to FIGS. 12 to 21 there is shown a sampler 200 according to a second embodiment of the invention that is intended for taking of blood samples in the 0.1 ml to 1.5 ml range. It is not limited to this range and may be made for smaller or larger sample sizes or ranges. The sampler 200 may be connected to a patient in a similar manner to that shown in FIG. 1.

As with the first embodiment the sampler comprises a hollow tubular (preferably cylindrical) barrel 202 and a complementary inner plunger 204.

The barrel 202 has first and second barrel sections, 206 and 208 respectively.

The first barrel section 206 is of a first diameter and provides a blood receiving volume 210.

The plunger 202 has a first plunger section 212 and a second plunger section 214. The first plunger section 212 is sized to fit within the first barrel section 206 and the second plunger section 214 is sized to fit within the second barrel section 208. The second plunger section 214 is sized to that a conventionally sized evacuated tube may be inserted into the interior of the second plunger section 214.

The first and second barrel sections 206 and 208 are disposed coaxially and spaced longitudinally relative to each other, with an annular flange 216 extending between an outer end 218 of the first section 206 and an inner end 220 of the second section 208.

The first barrel section 206 has a male Luer fitting 222 for connection to a female Luer fitting of a blood line at a first end 224. A one way valve 226 is located internally in the first section 206 at the first end 224. Two sealing rings 228 (there may be none, only one or more than two) are located on the interior of the first barrel section 206 near the junction of the first and second barrel sections 206, 208 and seal against the outer surface 238 of the first plunger section 212. The first barrel section 206 preferably has a groove 230 for locating each sealing ring. The sealing ring(s) 228 and any associated locating structure, such as groove(s) 230, are optional and may be omitted.

A gasket 232 is located on the inner end 234 of the first plunger section 212 and seals against the inner surface 236 of the first barrel section 206.

Insertion of the plunger 204 into the barrel 202 may be limited by one or both of the gasket 232 at first plunger section inner end 234 contacting the one way valve and the inner end 240 of the of the second plunger section 214 contacting the flange 216. Where the inner end 240 of the second plunger section 214 contacting the flange 216 limits insertion of the plunger 204 into the barrel, preferably there is minimal space between the gasket 232 and the one way valve 226. Preferably the gasket 232 and the one-way valve 226 have complementary male-female shaping to provide effectively no gap between their surfaces.

A cannula 244 is mounted in the plunger 204 and extends from the inner end 234 of the first plunger section, through the hollow interior 246 of the first plunger section 212, through the end 240 of the second plunger section 214 and into the interior 248 of the second plunger section 214, with the sharpened free end 250 of the cannula 244 located within the second plunger section 214 and facing the open end 242.

The region 252 of the junction of the second plunger section 214 with the first plunger section 212 preferably provides support for the cannula 244. In the embodiment shown a tubular portion 254 extends longitudinally both sides of the junction 252. In this embodiment the inner end 240 of the second section 214 is fully closed, so closing the outer end 256 of the first plunger portion 212. Thus, for manufacturing purposes, the inner end 234 of the first plunger section 212 is manufactured with an open end, to allow a component of the injection mould to define the hollow interior 246 and the portion of the tubular support 254 that extends into the hollow interior 246.

The gasket 232 mounted on the inner end 234 has an opening or passageway 256 through which the cannula 244 passes. Because the first plunger section 212 does not support the cannula at or near the plunger's inner end 234, support for the cannula free end is provided by the gasket 232. Accordingly, the gasket has a tubular section 258 that extends longitudinally within the section 212 along the cannula 244 to provide support. The tubular section 258 may be omitted.

As with the first embodiment the portion of the cannula 244 that extends into the second plunger section 214 is preferably sheathed with sheath 260.

Because the inner portion of the first barrel section 206 is sealed with the sealing rings 228 there is no need to seal the second plunger section 214 against the second barrel section 208 to prevent contamination of the interior of the first barrel section 206. Accordingly, there are no sealing rings in the second barrel section 208. Further, the second barrel section 208 may be shorter than the second plunger section 214.

A hinged closure 262 is provided on the end 264 of the second plunger section 214. The closure 262 of the plunger 204 is similar to that of the first embodiment and is preferably a transition fit in the end 264 of the second plunger section 214. However, because the second barrel section 208 is shorter than the second plunger section 214, the arm 266 of the closure 262 engages the second plunger section 214 rather than the second barrel section 208. If desired the two second sections 208, 214 may be of similar length with the arm 266 engaging the second barrel section 208 and securing the plunger at the fully inserted position. As an alternative, the second plunger section 214 may be provided with one or more separate members that engage the end of the second barrel section 208.

The second plunger section 214 and second barrel section 208 are provided with flanges 268 and 270 to aid the user in using the sampler. Because the ends of the two second sections are not adjacent to each other when the plunger 204 is fully inserted either or both of the flanges 268 and 270 may be asymmetric and may, for example, comprise two opposed finger flanges.

Because the second barrel and plunger sections 208, 214 do not need to seal against each other they need not circular in cross section (but preferably are) or be of constant cross section along their lengths (but may be). As best seen in FIGS. 17 to 19, the second barrel and plunger sections 208, 214 are tapered, with the inner ends 216, 240 being of smaller cross section than the respective outer ends 272, 242.

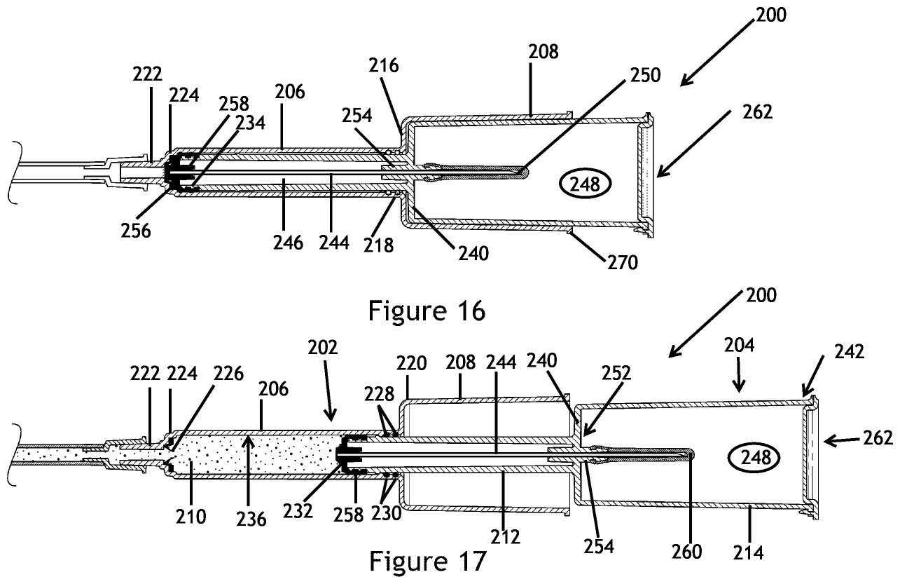

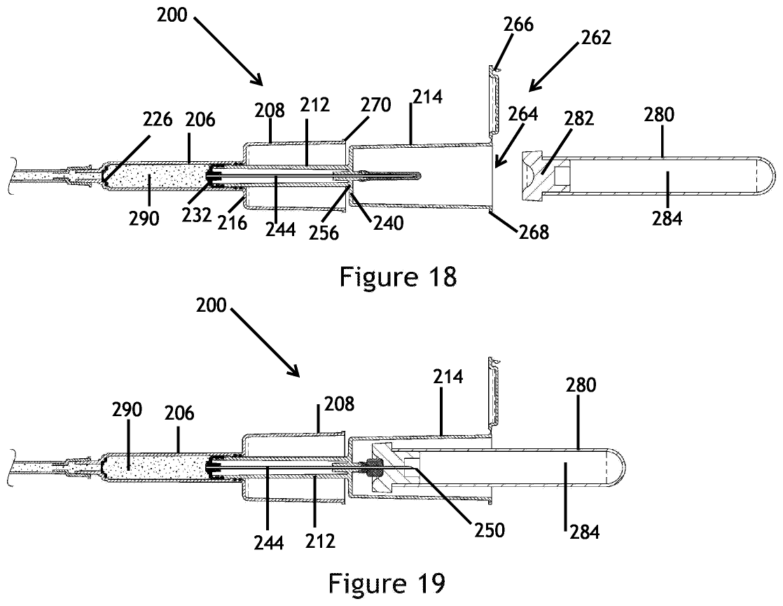

As with the first embodiment, the sampler 200 is supplied in the fully inserted position with the closure 262 in the closed position. After connection to the blood line, as in FIG. 16, the user grasps the barrel 202 and an exposed part of the second plunger section 214. The plunger 204 is withdrawn, opening one way valve 226 and drawing blood 290 into the interior 210 of first barrel section, as in FIG. 17, using the volume markings 274 to determine where to withdraw the plunger to. As with the first embodiment the one way valve 226 is normally closed and so closes when outward plunger movement ceases, as in FIG. 18. The closure 262 is opened, as in FIG. 18, an evacuated tube 280 is inserted into the second plunger section 214 and impaled on the cannula 244, as in FIG. 19. As with the first embodiment, pressing the evacuated tube into the plunger and on to the cannula cannot result in escape of any of the blood 290 in the barrel and blood can only leave the barrel once the free end 250 of the cannula has passed through the bung 282 and into the interior 284 of the evacuated tube.

As with the first embodiment the pressure difference across the one way valve 226 generated by the evacuated tube is not enough to open the valve 226. Once the free end 250 of the cannula 244 has passed through the bung 282 into the interior 284 of the evacuated tube 280 and the evacuated tube 280 is fully impaled, the plunger 204 is then inserted fully into the barrel 202, transferring blood from the interior 210 of barrel 202 into the interior 284 of the evacuated tube 280. As with the first embodiment the components may be configured such that the pressure difference initially applied by the evacuated tube 280 overcomes the resistance to movement of the plunger 208 relative to the barrel 206. As with the first embodiment the evacuated tube 280 is removed, the closure preferably closed and the process repeated as many times as needed.

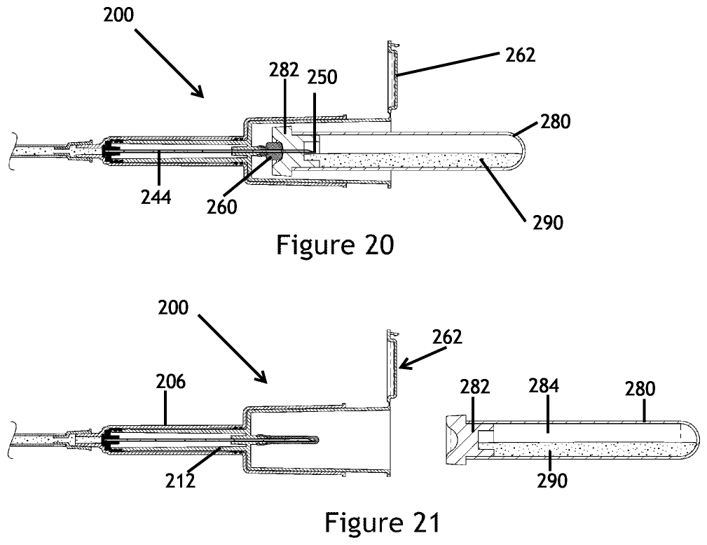

As best seen in FIGS. 20 and 21, after an evacuated tube 208 has been filled and withdrawn a very small amount of the blood 290 remains inside the cannula 244. This also applies to the first embodiment but this is less significant as in the first embodiment the barrel volumes are proportionally greater than the cannula volume. Since the cannula is initially blood free the first sample transferred to an evacuated tube is inherently smaller by the volume of the cannula. Subsequent samples are not impacted. The volume of the cannula is not considered significant but, if desired or necessary, may addressed by instructions and/or physical structure.

For both embodiments the one way valve may be duckbill (bi, tri or quad-cuspid) valve, dome valve or x-fragm valve, preferably a dome valve or x-fragm valve. The exact design is not critical to the invention.

The embodiments described have a gasket that extends over the inner end of the plunger and fully across the barrel interior. The invention is not limited to this gasket arrangement and other gasket arrangements may be utilised. For example, the inner end region of a plunger may be provided with one or more peripheral O-rings that seal against the barrel. Where the gasket does not extend over the inner end region of the plunger that inner end region may be shaped so that when the plunger is fully inserted into the barrel the effective internal volume is substantially or effectively zero. The gasket and/or an inner end portion of the plunger may engage the one way valve when the plunger is fully inserted into the barrel.

It will be appreciated that evacuated tubes are supplied with an internal pressure lower than ambient. The person skilled in the art can readily ascertain the relevant internal pressure or range of pressures of the evacuated tubes intended to be used with relevant samplers of the invention.

It will be appreciated that versions of the invention are not limited to the plunger alone providing support for the inner end of the cannula, as in the first embodiment, or the gasket alone providing support for the inner end of the cannula, as in the second embodiment. Support for the cannula at or near its the inner end may be provided by the plunger alone, the gasket alone or the plunger and the gasket.

Unless the context clearly requires otherwise, it is to be understood that, if any prior art is referred to herein, such reference does not constitute an admission that the prior art forms a part of the common general knowledge in the art, in Australia or any other country.

Unless the context clearly requires otherwise, throughout the description and any claims the words “comprise”, “comprising”, and the like are to be construed in an inclusive sense as opposed to an exclusive or exhaustive sense; that is to say, in the sense of “including, but not limited to”.

The features of the invention described or mentioned in this document may be combined in any combination of features where features are not mutually exclusive.

It will be apparent to those skilled in the art that many obvious modifications and variations may be made to the embodiments described herein without departing from the spirit or scope of the invention.

1. A sampler comprising:
a tubular barrel having a first barrel section with an interior closed at one end and an open at the other end;
a plunger received within the first barrel section for reciprocal movement therein via the open end and including a gasket that engages and seals against the first barrel section, thereby defining a fluid receiving volume between the closed end and the gasket;
an inlet in fluid communication with the fluid receiving volume via a flow path;
a one way valve located in the flow path and configured to allow fluid to flow from the inlet into the fluid receiving volume and to prevent fluid to flow out of the fluid receiving volume through the inlet;
the plunger including an outlet in fluid communication with the fluid receiving volume, the plunger operable to draw fluid into fluid receiving volume via the inlet and expel fluid from the fluid receiving volume via the outlet.

2. The sampler of claim 1 wherein the plunger comprises a tubular portion adapted to receive an evacuated sampling tube.

3. The sampler of claim 2 wherein the outlet extends into the tubular portion.

4. The sampler of claim 3 wherein at least part of the tubular portion is sized to fit within at least part of the first barrel section.

5. The sampler of claim 4 wherein the plunger comprises first and second plunger sections arranged end on end with the first plunger section nearer the closed end than the second plunger section.

6. The sampler of claim 5 wherein the second plunger section comprises the tubular portion.

7. The sampler of claim 6 wherein the barrel includes a second barrel section.

8. The sampler of claim 7 wherein at least part of the tubular portion is sized to fit within at least part of the second barrel section.

9. (canceled)

10. (canceled)

11. (canceled)

12. (canceled)

13. (canceled)

14. The sampler of claim 1 wherein the one way valve is normally closed and configured to open when a first predetermined pressure difference is applied across the one way valve.

15. The sampler of claim 14 wherein the first predetermined pressure difference is greater than that available from an evacuated tube.

16. The sampler of claim 14 configured such that the plunger is movable into the barrel when there is a second predetermined pressure difference between the fluid receiving volume and the outside environment.

17. The sampler of claim 16 wherein the second predetermined pressure difference is less than that available from an evacuated tube.

18. The sampler of claim 16 wherein the first predetermined pressure difference is greater than the second predetermined pressure difference.

19. The sampler of claim 1 wherein the inlet comprises an inlet tubular portion and the one way valve is mounted in the interior of the barrel at an inner end of the inlet tubular portion.

20. The sampler of claim 19 wherein when the plunger is fully inserted into the barrel the volume of the fluid receiving volume is substantially or effectively zero.

21. The sampler of claim 19 wherein the gasket and/or an inner end portion of the plunger engages the one way valve when the plunger is fully inserted into the barrel.

22. (canceled)

23. (canceled)

24. The sampler of claim 1 comprising two different sets of volume markings, with a first set for use when drawing a first sample of fluid into the sampler and the second set for use when drawing subsequent samples of fluid into the sampler.

25. (canceled)

26. A method of transferring at least one sample of fluid from a fluid source to at least one fluid container, the method comprising:
a. connecting the inlet of a sampler as claimed in any one of the previous claims to the fluid source;
b. increasing the effective volume of the fluid receiving volume and causing fluid to flow from the fluid source via the inlet into the fluid receiving volume;
c. connecting the outlet to the interior of a first fluid container;
d. causing the plunger to move relative to the first barrel section to decrease the effective volume of the fluid receiving volume and transferring a first sample of fluid in the fluid receiving volume via the outlet to the interior of the first fluid container, and
e. disconnecting the interior of the first fluid container from the outlet.

27. The method of claim 26 further comprising after step e and whilst the inlet remains connected to the fluid source,
f. increasing the effective volume of the fluid receiving volume and causing fluid to flow from the fluid source via the inlet into the fluid receiving volume;
g. connecting the interior of a subsequent fluid container to the outlet;
h. causing the plunger to move relative to the first barrel section to decrease the effective volume of the fluid receiving volume and transferring a subsequent sample of fluid in the fluid receiving volume via the outlet to the interior of the subsequent fluid container, and
i. disconnecting the interior of the subsequent fluid container from the outlet.

28. The method of claim 27 wherein moving the plunger relative to the first barrel section to decrease the effective volume of the fluid receiving volume is at least initiated by application to the outlet of a pressure that is lower than ambient pressure.
